(19) United States
(12) Patent Application Publication
(10) Pub. No.: US 2021/0072161 A1
(43) Pub. Date: Mar. 11, 2021

(54) DETECTING DIESEL EXHAUST FLUID IN FUEL SYSTEM ICING INHIBITOR

(71) Applicant: EMCEE ELECTRONICS, INC., Venice, FL (US)

(72) Inventor: Ryan Matthew Case, Sarasota, FL (US)

(21) Appl. No.: 17/012,261

(22) Filed: Sep. 4, 2020

Related U.S. Application Data
(60) Provisional application No. 62/896,844, filed on Sep. 6, 2019.

(51) Int. Cl. G01N 21/78

(52) U.S. Cl. CPC G01N 21/78

(57) ABSTRACT

Ways of detecting an aqueous solution in a glycol ether are provided, including detecting an aqueous solution, such as diesel exhaust fluid (DEF) in fuel system icing inhibitor (FSII). A metal salt and the glycol ether are combined, and the glycol ether is identified as including the aqueous solution when the combined metal salt and the glycol ether result in a color change. The presence of the aqueous solution, including the presence of ammonia derived from urea, can indicate contamination of FSII with water and/or DEF and identify the container or reservoir of FSII as unsuitable for addition to aviation fuel.

### CROSS-REFERENCE TO RELATED APPLICATIONS

This application claims the benefit of U.S. Provisional Application No. 62/896,844, filed on Sep. 6, 2019. The entire disclosure of the above application is incorporated herein by reference.

### FIELD

The present technology relates to detecting a presence of an aqueous additive in an organic liquid, including the detection of diesel exhaust fluid in fuel system icing inhibitor.

### INTRODUCTION

This section provides background information related to the present disclosure which is not necessarily prior art.

Aircraft fueling can involve various ways of storing, transporting, and dispensing aviation fuel. Handling and use of aviation fuel can include the addition of one or more various aviation fuel additives, such as fuel system icing inhibitor (FSII). Likewise, handling and use of aviation fuel can include equipment using other types of fuel and other types of additives. For example, an aviation fuel transport vehicle, such as a tanker truck, can be powered by a diesel engine system that includes a reservoir for the Environmental Protection Agency (EPA) mandated additive diesel exhaust fluid (DEF).

Addition of FSII to aviation fuel can mitigate performance issues arising from water contamination. Despite stringent measures put in place to eliminate water from aviation fuel, the possibility of water contamination still exists. For example, aviation fuel can contain a small amount of dissolved water that does not appear in droplet form. Water present in aviation fuel can subsequently form ice crystals at high altitudes and/or low temperatures that can block fuel filters, injectors, and/or fuel lines of an aircraft. FSII can be added to aviation fuel to prevent the formation of such ice crystals, providing additional security in the event of water contamination in aviation fuel. FSII is a mandatory additive in many military aviation fuel specifications, for example. FSII dissolves sparingly in aviation fuel, but more easily in water, where water present in the aviation fuel will extract FSII from the fuel. The FSII thereby acts to lower the freezing point of the water (e.g., FSII can depress the freezing point of water to −43° C.) and therefore prevent the formation of ice crystals that could block the fuel supply system of the aircraft. FSII is sometimes referred to by the trademark Prist. Chemically, FSII can be almost pure (99.9%) ethylene glycol monomethyl ether (EGMME, 2-methoxy ethanol, APISOLVE 76, CAS number 109-86-4); or since 1994, diethylene glycol monomethyl ether (DEGMME, 2-(2-methoxy ethoxy) ethanol, APITOL 120, methyl carbitol, CAS number 111-77-3), which has a higher flash point. FSII can be added to aviation fuel as the fuel is dispensed into an aircraft. The mixture of FSII and aviation fuel can have between 0.10% and 0.15% by volume FSII. For optimal performance, the FSII can be distributed evenly throughout the aviation fuel as it is being dispensed to the aircraft. Simply adding FSII after the aircraft is fueled may not be sufficient.

A diesel engine powered tanker truck can require addition of DEF to minimize exhaust emissions. DEF can be formulated, for example, as an aqueous urea solution of 32.5% urea and 67.5% deionized water. DEF has been standardized as AUS 32 (aqueous urea solution) in ISO 22241. The DEF additive is consumed in selective catalytic reduction to lower NOx concentration in diesel engine exhaust emissions. For example, diesel engines can be run with a lean burn air-to-fuel ratio (overstoichiometric ratio), to ensure the full combustion of soot and to prevent exhausting of unburnt fuel. Excess air, however, can lead to the generation of nitrogen oxides (NOx) from the nitrogen in the air, where these nitrogen oxides are undesirable pollutants. Selective catalytic reduction (SCR) reduces the amount of NOx released into the atmosphere, where DEF is injected into the exhaust, resulting in vaporization and decomposition of the aqueous urea to form ammonia and carbon dioxide. Within the SCR catalyst, NOx is catalytically reduced by the ammonia into harmless water and nitrogen, which are released through the exhaust.

An aircraft refueling facility can therefore have onsite various reservoirs or containers of aviation fuel and FSII as well as various reservoirs or containers of diesel fuel and DEF. FSII is clear and can have the appearance of water. Likewise, DEF is clear and can have the appearance of water. Accordingly, opportunity exists for mistakenly adding DEF to a reservoir or container of FSII or using DEF in place of FSII, where DEF could then find its way into aviation fuel onboard an aircraft. The result of DEF or a mixture of DEF/FSII in aviation fuel can be the catastrophic failure of a jet engine of the aircraft. A need therefore exists to determine if FSII is free of DEF prior to addition of FSII to aviation fuel.

### SUMMARY

The present technology includes processes and articles of manufacture that relate to detection of an aqueous solution in a glycol ether, such as the detection of diesel exhaust fluid in fuel system icing inhibitor.

Detection of an aqueous solution in a glycol ether can include combining a metal salt and the glycol ether. The glycol ether can be identified as including the aqueous solution when the combined metal salt and the glycol ether result in a color change. The metal salt can be provided in a substantially anhydrous form and the metal salt can include a metal that can complex with ammonia. The metal salt can include copper sulfate. The glycol ether can include ethylene glycol monomethyl ether and/or diethylene glycol monomethyl ether. Particular embodiments can detect an aqueous solution in fuel system icing inhibitor (FSII) by combining substantially anhydrous copper sulfate and the FSII. The FSII can be identified as including the aqueous solution when the combined metal salt and the glycol ether result in a color change. The FSII can be identified as including water when the color change results in a visible blue color. And the FSII can be identified as including diesel exhaust fluid (DEF) when the color change results in a visible blue-green color.

Articles of manufacture can include various kits for detecting DEF in FSII. Such kits can include substantially anhydrous copper sulfate and a sample container for holding a sample of FSII, where at least a portion of the sample container is substantially transparent. The substantially anhydrous copper sulfate can be sealed in the sample container. Alternatively, the substantially anhydrous copper sulfate can be sealed in a moisture resistant packet. The moisture resistant packet can be opened and the substantially anhydrous copper sulfate can be added to an aliquot of FSII within the sample container. Color change can be monitored through the portion of the sample container that is substantially transparent. For example, the sample container can comprise a clear vial with a removable cap.

Further areas of applicability will become apparent from the description provided herein. The description and specific examples in this summary are intended for purposes of illustration only and are not intended to limit the scope of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology provides ways to determine if glycol ethers, such as fuel system icing inhibitor (FSII), are free of aqueous solutions, such as diesel exhaust fluid (DEF). It can be important to ascertain if a reservoir or container of FSII has been inadvertently contaminated with DEF prior to dispensing the FSII into aviation fuel in an aircraft refueling operation. In particular, an aircraft refueling facility can have onsite various reservoirs and containers of aviation fuel and FSII as well as various reservoirs and containers of diesel fuel and DEF, where the aviation fuel and FSII are destined for aircraft and the diesel fuel and DEF are destined for transport vehicles, such as a tanker truck. However, FSII is clear and has the appearance of water, where DEF is likewise clear and also has the appearance of water. Accordingly, opportunity exists for mistakenly adding DEF to a reservoir or container of FSII or using DEF in place of FSII, where DEF could then find its way into aviation fuel onboard an aircraft. The result of DEF or a mixture of DEF/FSII in aviation fuel can be the catastrophic failure of a jet engine of the aircraft. The present technology can hence rapidly detect DEF in FSII prior to injection in aviation fuel, providing an integrity assessment of the FSII at the point of use. In this way, the present technology can militate against the misapplication of additives at an aircraft fueling site.

Methods of detecting an aqueous solution in a glycol ether are provided. Such methods can include combining a metal salt and the glycol ether and identifying the glycol ether as including the aqueous solution when the combined metal salt and the glycol ether result in a color change. Combining the metal salt and the glycol ether can be accomplished by adding one of the metal salt and the glycol ether to the other one of the metal salt and the glycol ether. For example, the glycol ether can be an aliquot of FSII within a sample container having a clear portion to which the metal salt in power form is added. The sample container having the aliquot of FSII and the powered metal salt can be capped and mixed, where the color change (if present) can be observed through the clear or transparent portion of the container.

The metal salt can include the following aspects. The metal salt can be substantially anhydrous. That is, it can be very difficult to achieve a metal salt completely free of water, but the metal salt can be free of water as practicable using known laboratory methods and considered anhydrous. The metal salt can therefore be maintained in a substantially anhydrous state by storage within or containment by a moisture resistant barrier; e.g., a sealed foil packet. The metal salt can also be contained within an anhydrous environment, such as a desiccator, or packaged or contained with one or more desiccants; e.g., silica gel. The metal salt can react with water. For example, dissociation of the metal salt in the presence of an aqueous solution can result in the metal cation reacting with water of the aqueous solution and exhibiting a certain color change. This can happen when the glycol ether has been adulterated with an aqueous solution. The metal salt can include a metal that can complex with ammonia. For example, dissociation of the metal salt in the presence of an aqueous solution can result in the metal cation complexing with ammonia and exhibiting a certain color change. This can happen when the glycol ether has been adulterated with DEF. Examples of metal salts that can complex with ammonia include copper salts, nickel salts, cobalt salts, and combinations thereof. Where the metal salt includes a copper salt, the metal salt can comprise copper sulfate, and more particularly, substantially anhydrous copper sulfate.

As referred to herein, copper sulfate includes copper(II) sulfate, also known as cupric sulfate, copper(II) sulphate, and cupric sulphate. Copper sulfate can be represented by the chemical formula: CuSO₄(H₂O)ₓ, where x can range from 0 to 5. Substantially anhydrous copper sulfate can be represented by the chemical formula: CuSO₄(H₂O)₀₋₁, where the average number of water (H₂O) molecules associated with the copper sulfate can range from 0 to 1. In certain embodiments, substantially anhydrous copper sulfate can be represented by the chemical formula: CuSO₄(H₂O)_{0-0.25}, where the average number of water (H₂O) molecules associated with the copper sulfate can range from 0 to 0.25. Still further embodiments include where substantially anhydrous copper sulfate can be represented by the chemical formula: CuSO₄(H₂O)_{0-0.1}, where the average number of water (H₂O) molecules associated with the copper sulfate can range from 0 to 0.1. Substantially anhydrous copper sulfate is substantially white or off-white/grey in color, which changes toward a blue color as copper sulfate is progressively hydrated until the copper sulfate reaches the pentahydrate form: CuSO₄(H₂O)₅.

The glycol ether can include the following aspects. The glycol ether can include one or more of: ethylene glycol monomethyl ether; ethylene glycol monoethyl ether; ethylene glycol monopropyl ether; ethylene glycol monoisopropyl ether; ethylene glycol monobutyl ether; ethylene glycol monophenyl ether; ethylene glycol monobenzyl ether; propylene glycol methyl ether; diethylene glycol monomethyl ether; diethylene glycol monoethyl ether; diethylene glycol mono-n-butyl ether; and dipropyleneglycol methyl ether. In certain embodiments, the glycol ether includes ethylene glycol monomethyl ether. In other embodiments, the glycol ether includes diethylene glycol monomethyl ether. In still further embodiments, the glycol ether includes FSII.

Combination of the metal salt and the glycol ether can be effected in various ways. As noted herein, one of the metal salt and the glycol ether can be added to the other one of the metal salt and the glycol ether. In certain embodiments, the metal salt is added to the glycol ether; e.g., substantially anhydrous copper sulfate in powder form is added to an aliquot of FSII. Once combined, the metal salt and the glycol ether can be mixed by inversion, manual or mechanical shaking, swirling, use of a vortex mixer, etc. The mixing of metal salt and glycol ether can be done for seconds or minutes; e.g., from 30 seconds to 5 minutes. The mixing can also be brief (e.g., 30 seconds) and the mixture allowed to sit for a time period (e.g., 5 minutes). Depending on the presence and amount of aqueous solution within the glycol ether, the resulting color change can occur within seconds or may require a several minutes to become visible. The color change, if present, is typically visible by 5 minutes or less.

Certain methods of detecting an aqueous solution in fuel system icing inhibitor (FSII) are provided. Such methods can include combining substantially anhydrous copper sulfate and the FSII and identifying the FSII as including the aqueous solution when the combined metal salt and the glycol ether result in a color change. Such methods can further include identifying the FSII as including water when the color change results in a visible blue color or identifying the FSII as including diesel exhaust fluid (DEF) when the color change results in a visible blue-green color. That is, the copper cation of the copper sulfate can react with water to produce the visible blue color and the copper cation can further react with urea (from DEF, for example) to break down the urea into ammonia and carbon dioxide, where the copper cation can complex with the ammonia to produce the visible blue-green color.

Kits for detecting an aqueous solution in a glycol ether are provided, including kits for detecting diesel exhaust fluid (DEF) in fuel system icing inhibitor (FSII). Such kits can include a metal salt and a sample container for holding a sample of the glycol ether, where at least a portion of the sample container is substantially transparent. Certain embodiments include substantially anhydrous copper sulfate and a sample container for holding a sample of FSII, wherein at least a portion of the sample container is substantially transparent. The substantially anhydrous copper sulfate can be sealed in the sample container or the substantially anhydrous copper sulfate can be sealed in another way, such as within a moisture resistant container or packet. The sample container can comprise a clear vial with a removable cap.

Use of the kits provided herein can include adding an amount of the metal salt to the glycol ether. For example, adding substantially anhydrous copper sulfate to an aliquot of FSII in a container and shaking results in a creamy white color from a suspension of the metal salt within the FSII. If the mixture stays the same or clears up slightly, there is little or no presence of water or DEF. If the mixture turns blue in color, then there is water present in the FSII aliquot. If the mixture turns blue-green in color, then there is DEF in the FSII aliquot. The metal salt can be provided in powder form in a moisture resistant packet, where the packet can include an opening mechanism, such as a tear line, tear strip, notch, or perforated portion. The packet can be kept sealed until the test is ready to be conducted. In this way, the metal salt (in anhydrous form) can be kept substantially free of moisture, including exposure to environmental water vapor such as humidity in the ambient air, as the metal salt may be hygroscopic.

Other embodiments of the present technology include where the metal salt comprises substantially anhydrous copper chloride, which has a reddish-brown color that further changes in the presence of water as well as aqueous urea. The metal salt can include other copper salts, such as copper chloride, copper bromide, copper nitrate, copper acetate, and mixtures thereof. However, certain substantially anhydrous copper salts (e.g., copper chloride) are not white or off-white/grey in color and it can be more difficult to therefore observe a visible color change in the glycol ether sample when water or aqueous urea is present. As such, the use of substantially anhydrous copper sulfate can be preferred in certain embodiments as it provides an readily observable colorimetric change when contacting water or aqueous urea.

Metal salts including metal other than copper can be included. For example, nickel sulfate (e.g., substantially anhydrous nickel sulfate) can be used. Handling and/or disposal of certain metal salts, however, can present an issue such that it can be preferable to use metal salts that minimize any such hazards or toxicity; e.g., copper salts versus nickel salts. Nickel sulfate, for example, is classified as a carcinogen and use thereof can complicate handling and/or disposal. Further examples of metal salts include cobalt salts, where cobalt(III) can complex with ammonia and provide a color change in the presence of aqueous urea. Accordingly, the metal salt used in the present technology can be characterized as having a metal that can complex with water and that can complex with ammonia.

Various embodiments of kits used to practice the methods provided herein include a metal salt, where the metal salt can be substantially anhydrous (e.g., substantially anhydrous copper sulfate) and a sample container for holding a sample of glycol ether (e.g., FSII), where at least a portion of the sample container is substantially transparent. The sample container can be configured as a clear vial (e.g., glass vial) capable of holding an aliquot of the glycol ether to be tested (e.g., about 2 mL of FSII). An aliquot of the glycol ether can withdrawn or dispensed from a reservoir or container of the glycol ether and added to the sample container. A moisture resistant packet containing metal salt in powder form can be opened and the powdered metal salt added to the aliquot of the glycol ether in the sample container. The sample container can be capped or sealed in some manner and the metal salt and glycol ether mixed by shaking, for example. The metal salt powder can be added relatively quickly after opening the moisture resistant packet to minimize exposure of the metal salt powder to humidity in the air. After mixing, presence of a color change can be assessed from seconds up to five minutes, or so. For example, very little contamination can produce a slight color change in about 5 minutes, whereas 5% to 10% contamination can result in a rapid color change. If a sample of FSII results in a color change, indicating that DEF contamination may be present, the FSII can rejected for use in aircraft refueling and can be quarantined for further testing or disposed of, where a new reservoir or container of FSII can be provided, tested for DEF, and upon passing the test, dispensed into aviation fuel when refueling an aircraft.

Several benefits and advantages can result from the present technology. In particular, the methods and kits described herein provide the only known rapid, visible test for ascertaining DEF contamination of FSII. Detecting such undesired contamination can be very important as FSII is added to jet fuel used in virtually all military fuels and by request in commercial aviation. Visually, there is no way to determine if FSII has been mixed with DEF or replace with DEF, as both FSII and DEF are clear and appear similar to water. Thus, the present technology provides a rapid, visible test to detect contamination of FSII with DEF in locations, such as airports, that keep reservoirs or containers of both FSII and DEF onsite.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A method of detecting an aqueous solution in a glycol ether, the method comprising:
combining a metal salt and the glycol ether;
identifying the glycol ether as including the aqueous solution when the combined metal salt and the glycol ether result in a color change.

2. The method of claim 1, wherein the metal salt is substantially anhydrous.

3. The method of claim 1, wherein the metal salt includes a metal that can complex with ammonia.

4. The method of claim 1, wherein the metal salt includes a member of a group consisting of a copper salt, a nickel salt, a cobalt salt, and combinations thereof.

5. The method of claim 1, wherein the metal salt includes a copper salt.

6. The method of claim 1, wherein the metal salt includes copper sulfate.

7. The method of claim 1, wherein the metal salt includes substantially anhydrous copper sulfate.

8. The method of claim 1, wherein the glycol ether is a member of a group consisting of:
ethylene glycol monomethyl ether; ethylene glycol monoethyl ether; ethylene glycol monopropyl ether; ethylene glycol monoisopropyl ether; ethylene glycol monobutyl ether; ethylene glycol monophenyl ether; ethylene glycol monobenzyl ether; propylene glycol methyl ether; diethylene glycol monomethyl ether; diethylene glycol monoethyl ether; diethylene glycol mono-n-butyl ether; dipropyleneglycol methyl ether; and combinations thereof.

9. The method of claim 1, wherein the glycol ether includes ethylene glycol monomethyl ether.

10. The method of claim 1, wherein the glycol ether includes diethylene glycol monomethyl ether.

11. The method of claim 1, wherein the glycol ether includes fuel system icing inhibitor.

12. A method of detecting an aqueous solution in fuel system icing inhibitor (F SIT), the method comprising:
combining substantially anhydrous copper sulfate and the FSII;
identifying the FSII as including the aqueous solution when the combined metal salt and the glycol ether result in a color change.

13. The method of claim 12, further comprising identifying the FSII as including water when the color change results in a visible blue color.

14. The method of claim 12, further comprising identifying the FSII as including diesel exhaust fluid (DEF) when the color change results in a visible blue-green color.

15. A kit for detecting diesel exhaust fluid (DEF) in fuel system icing inhibitor (FSII), the kit comprising:
substantially anhydrous copper sulfate; and
a sample container for holding a sample of FSII, wherein at least a portion of the sample container is substantially transparent.

16. The kit of claim 15, wherein the substantially anhydrous copper sulfate is sealed in the sample container.

17. The kit of claim 15, wherein the substantially anhydrous copper sulfate is sealed in a moisture resistant packet.

18. The kit of claim 15, wherein the sample container comprises a clear vial with a removable cap.
